(19) United States
(12) Patent Application Publication
(10) Pub. No.: US 2013/0211502 A1
(43) Pub. Date: Aug. 15, 2013

(54) METHOD OF USING AMNION ALLOGRAFT IN CORONARY ARTERY BYPASS GRAFTING

(71) Applicant: AFCELL MEDICAL, (US)

(72) Inventor: Robin R. YOUNG, Wayne, PA (US)

(73) Assignee: AFCELL MEDICAL, Parsippany, NJ (US)

(21) Appl. No.: 13/767,204

(22) Filed: Feb. 14, 2013

Related U.S. Application Data
(60) Provisional application No. 61/598,421, filed on Feb. 14, 2012.

(51) Int. Cl. A61F 2/06

(52) U.S. Cl. CPC A61F 2/06
USPC 623/1.23

(57) ABSTRACT

Improved methods for coronary artery bypass grafting surgeries are described. The methods utilize an allograft comprising a layer of amnion to improve the performance and reduce complications of the surgeries and the allograft has a pre-made size and shape suitable for the application.

### CROSS-REFERENCE TO RELATED APPLICATIONS

This application is entitled to priority pursuant to 35 U.S.C. §119(e) to U.S. Provisional Patent Application No. 61/598,421, filed Feb. 14, 2012, which is hereby incorporated by reference herein in its entirety.

### BACKGROUND OF THE INVENTION

Coronary artery bypass grafting (CABG) is one of the most commonly performed major operations in the U.S. It is advised for patients with significant atherosclerotic narrowing and blockage of the heart arteries. Arteries or veins from elsewhere in the patient's body are grafted to the coronary arteries to re-route the blood around the blockages in the coronary arteries, allowing improved blood flow to deliver sufficient oxygen and nutrients to the myocardium (heart muscle) to thus prevent a heart attack or sudden death. Conventionally, an artery from behind the breast bone, or veins from the legs are used to “bypass” the blood around the coronary artery blockages. This surgery is usually performed with the heart stopped by using cardiopulmonary bypass (the heart-lung machine). The operation can also be performed on a beating heart, without using the heart-lung machine, so-called “off-pump” surgery.

Overall mortality related to CABG is 3-4%. During and shortly after CABG surgery, heart attacks occur in 5 to 10% of patients and are the main cause of death. About 5% of patients require exploration because of bleeding. This second surgery increases the risk of chest infection and lung complications. Stroke occurs in 1-2%, primarily in elderly patients. There is a need to improve the wound healing and reduce scar formation to thus improve the performance and reduce complications of CABG.

The amnion is a thin, cellular, extraembryonic membrane that forms the inner membrane of a closed sac surrounding and protecting an embryo in reptiles, birds, and mammals. The sac contains the fetus and amniotic fluid or liquor amnii, in which the embryo is immersed, nourished and protected. Typically, the amnion is a tough, transparent, nerve-free, and nonvascular membrane consisting of two layers of cells: an inner, single-cell-thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid. The amnion has multiple functions, i.e., as a covering epithelium, as an active secretary epithelium, and for intense intercellular and transcellular transport.

Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side.

The fetal membrane including amnion and chorion has been used in surgeries documented as early as 1910. See Trelford and Trelford-Sauder, The Amnion in Surgery, Past and Present, 134 AM J. OBSTET. GYNECOL 833 (1979). Amnioplastin, an isolated and chemically processed amniotic membrane, was used for continual dural repair, peripheral nerve injuries, conjunctival graft and flexor and tendon repair. See e.g., Chao et al., “A New Method of Preventing Adhesions: the Use of Amnioplastin after Craniotomy,” The British Medical Journal, Mar. 30, 1940. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery. In 1962, the fetal membrane was used to treat pelvic basins after total exenteration in dogs, however, trials in human proved disappointing.

In recent years, there have been renewed interests in the application of amnion in ocular surface reconstruction, for example, as an allograph for repairing corneal defects. See, for example, Tsai and Tseng, Cornea. 1994 Sep; 13 (5):389-400; and Dua et al., Br. J. Ophthalmol 1999, 83:748-752. In addition, amnion and amniotic fluid have recently been used as sources of placental stem cells. See, e.g., U.S. Pat. No. 7,255,879 and WO 200073421.

It is now discovered that using an allograft comprising a layer of amnion in CABG as described in the present invention significantly reduces inflammation and tissue adhesion, promotes uniform re-growth and epithelialization, prevents scar tissue formation, thus significantly improves performance and reduces complications of CABG.

### BRIEF SUMMARY OF THE INVENTION

In one general aspect, the present invention relates to a method of improving a coronary artery bypass grafting surgery. The improvement comprises applying at least one of an amniotic fluid and an allograft comprising a layer of amnion over a suture line or incision, or an otherwise damaged tissue site resulting from the coronary artery bypass grafting surgery, or over or under the pericardium membrane of the subject, wherein the allograft has a pre-made size and shape suitable for the application.

In another general aspect, the improvement comprises applying an allograft comprising a layer of amnion over a harvest site for a bypass vessel graft.

In yet another general aspect, the present invention relates to a kit comprising a plurality of allografts and instructions on how to use the allografts to improve a coronary artery bypass grafting surgery, wherein each of the plurality of allografts comprises a layer of amnion of a premade size and shape suitable for covering a suture line, an incision, or an otherwise damaged tissue site resulting from the coronary artery bypass grafting surgery, or for covering over or under the pericardium membrane of the subject.

Other aspects, features and advantages of the invention will be apparent from the following disclosure, including the detailed description of the invention and its preferred embodiments and the appended claims.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must be noted that as used herein and in the appended claims, the singular forms “a,” “an,” and “the” include plural reference unless the context clearly dictates otherwise.

Amnion has a complete lack of surface antigens, thus does not induce an immune response when implanted into a ‘foreign’ body, which is in contrast to most other allograft implants. Amnion also markedly suppresses the expression of the pro-inflammatory cytokines, IL-1α and IL-1β (Solomon et al., 2001, Br J Ophthalmol. 85 (4):444-9) and produces natural inhibitors of matrix metalloproteases (MMPs) expressed by infiltrating polymorphonuclear cells and macrophages. Hao et al., 2000, Cornea, 19 (3):348-52; Kim et al., 2000, Exp Eye Res. 70 (3):329-37). Amnion also down-regulates TGF-β and its receptor expression by fibroblasts leading to the ability to modulate the healing of a wound by promoting tissue reconstruction. Furthermore, amnion contains antimicrobial compounds with broad spectrum activity against bacteria, fungi, protozoa, and viruses for reduced risk of post-operative infection. All of these characteristics of amnion make it a potential allograft candidate to be used in coronary artery bypass grafting (CABG).

According to embodiments of the present invention, a coronary artery bypass grafting (CABG) can be conducted using any method known to those skilled in the art, such as, traditional CABG, off-pump CABG, minimally invasive direct CABG, etc. The improvement to the CABG according to the present invention comprises applying an allograft comprising a layer of amnion over a suture line or an incision resulting from the CABG, or over or under the pericardium membrane of the subject, wherein the allograft has a pre-made size and shape suitable for the application. The improvement can also comprise applying an allograft comprising a layer of amnion over a harvest site for a bypass vessel graft.

In one embodiment of the present invention, an allograft comprising a layer of amnion is used to improve the performance of a traditional CABG as described in detail in the following. The allograft can also be used in other CABG in similar manner in view of the present disclosure.

After the patient is brought to the operating room and moved onto the operating table, he is first rendered unconscious by general anesthesia. Patients are completely asleep during the entire course of the operation. The surgeon opens the patient's chest by dividing the breast bone or sternum via a median sternotomy, thus exposing the heart. The surgeon examines the heart for blockages and determines the number of bypasses to be performed. Up to four major blocked coronary arteries can be bypassed during one surgery.

As the surgeon opens and examines the heart, physician assistants harvest vessels, such as internal thoracic arteries, radial arteries and saphenous veins from the leg of the patient and prepare the vessels as bypass grafts. For example, physician assistants can remove the saphenous vein through incisions in the legs. The length of the incision is dependent upon the amount of vein required to complete the necessary number of “bypasses”. After the vein has been removed from the leg, it has the appearance of a long tube or “conduit”. It can be divided into separate shorter segments, each of which can be used for individual bypasses. The bypass graft can also be an artery, e.g., the left internal mammary artery (LIMA). During the operation, LIMA is taken down and one end is prepared for bypass grafting. When the harvesting of bypass grafts is completed, the patient is given heparin to prevent the blood from clotting.

According to one embodiment of the present invention, an allograft comprising a layer of amnion is placed over a harvest site for a bypass vessel graft. For example, the allograft can be placed over a LIMA harvest site or a saphenous vein harvest site. The allograft can be attached to the harvest site with a suture. The allograft can be of any size suitable for covering the sutures or other type of tissue injuries at the harvest site. For example, the allograft can have a rectangular shape of about 10 cm by 5 cm when used to cover a saphenous vein harvest site.

Preferably, a relatively thick layer of allograft is used to cover the harvest sites. In one embodiment of the invention, the allograft has a thickness of about 2 mm to 4 mm. It can have multiple layers of amnion or a combination of multiple layers of amnion and chorion.

Tubes or cannulae are inserted into the heart and major blood vessels surrounding the heart in preparation for cardiopulmonary (heart-lung) bypass. The patient can be placed on a heart-lung machine. Blood can be re-directed from the heart into the heart-lung machine. This permits the surgeon to safely operate on the still heart without blood pumping through it. The surgeon places the aortic cross-clamp across the aorta (the main artery leaving the heart). The heart can be stopped by cardioplegia, and the heart-lung machine continues to pump freshly oxygenated blood to the rest of the body, in effect, taking over the roles of the heart and lungs. However, operations without the use of heart-lung machines are also possible as known to those skilled in the art.

Routinely, the LIMA is used as a graft vessel. Saphenous vein from the leg is also routinely used for bypasses, particularly for additional blockages in coronary arteries. The coronary arteries are opened beyond the sites of the blockage, and the open ends of the LIMA and vein grafts are sewn to the openings in the coronary arteries. As soon as the open end of the LIMA is sewn to the coronary arteries, blood flow is established to that region of the heart. The surgeon sews one end of each vein graft onto the coronary arteries beyond the blockages and the other end to the aorta. After each end of a vein graft is sewn to the coronary arteries and the aorta, respectively, blood flow is established by the vein graft beyond the blocked arteries.

When all blockages are bypassed by bypass grafts, blood flow is established beyond all the blocked arteries, and the heart has effectively been “bypassed” The heart-lung machine is then gradually weaned off, and the patient's heart and lungs resume their normal functions and blood flow to the heart is restored. Usually, the heart starts beating again on its own. In some cases, mild electric shocks are used to restart the heart. In some cases, the aorta is partially occluded by a C-shaped clamp, the heart is restarted and suturing of the grafts to the aorta is done in this partially occluded section of the aorta while the heart is beating. Protamine is given to reverse the effects of heparin. The cannulae are removed from in and around the heart.

After the bypass grafts are sutured in, the pericardial cavity is washed with a saline solution containing one or more anti-microbial agents, such as gentamycin. An allograft comprising a layer of amnion is placed over the suture line or incision resulting from the surgery. It can be placed adjacent to the pericardium, e.g., along the anatomical planes. For example, the allograft is placed over the suture lines under the inverted Y-shaped incision of the pericardium to form a cover and barrier over the heart muscle and aorta. The allograft can be placed inside and/or outside of the pericardium, i.e., over or under the pericardium membrane. More than one allograft can be applied to cover different areas of the surgical wound, as appropriate. An amniotic fluid can also be applied to a suture line, incision or other damaged tissues resulting from the surgery.

In an embodiment of the present invention, the allograft is attached to a tissue, such as the pericardium membrane, with a suture, such as a 4.0 suture. Preferably, the allograft is able to hold a 4.0 polypropylene or monocryl suture.

The appropriate shape and dimension of the allograft are chosen based on the shape and size of the suture and incision. For example, the allograft can have an oval shape, about 3 cm-9 cm in length and about 2 cm-6 cm in width.

Preferably, the allograft placed adjacent to the pericardium is thin. In one embodiment of the invention, the allograft has a thickness of about 0.02 mm to 0.10 mm. It can have of a single layer of amnion, two layers of amnion, a layer of amnion and a layer of chorion, or a layer of amnion and a layer of other collagen membranes of biological origin. When the allograft is a combination of one or more layers of amnion and one or more layers of chorion, the layers can be arranged in any order. The multiple layers in the allograft can be subjected to a cross-linking treatment to make the layers closely adhere to each other in an integrated form.

In one embodiment of the present invention, the allograft can carry one or more therapeutic agents, such as anti-microbial agents, growth enhancing agent, anti-inflammatory agent, agents that prevent scarring, adhesions and tethering of internal organs and the heart, etc., to further improve the performance and reduce the complications of CABG. Examples of the growth enhancing agent include, but are not limited to, growth hormone, insulin like growth factor I, keratinocyte growth factor, fibroblast growth factor, epidermal growth factor, platelet derived growth factor and transforming growth factor, and a combination of any of the foregoing.

The two surfaces of human amnion are structurally different. The surface facing the fetus is smooth and hardly cell adhesive, comprising a thin layer of fine fibers. The surface facing the chorion is rough and suitable for cell proliferation, comprising thick fasciculus. In one embodiment of the present invention, the allograft is placed adjacent to the pericardium so that the chorion facing surface of the amnion faces the suture lines. In another embodiment of the present invention, the allograft is placed adjacent to the pericardium so that the fetus facing surface of the amnion faces the suture lines. The surgeon is provided with a range of sizes and shapes of allograft, such as the diamond shape, the curved cup shape, etc., which can be chosen and oriented according to the size and shape of the patient's anatomy.

After the allograft is placed in place, the sternum is wired together and the incisions are sutured closed. Drainage catheters are placed around the heart, which are usually removed 24 hours after the surgery. Temporary pacing wires to regulate the patient's heart rate are sewn to the surface of the heart, which are often removed before the patient goes home.

The patient is transported to the Cardiac Post-Anesthesia Care Unit, or an otherwise named specialized intensive care unit (ICU) caring exclusively for open-heart surgery patients. Patients generally awaken from anesthesia 4-6 hr after the operation. The following day all drainage catheters and monitoring lines are usually removed, and patients are transferred to the cardiac surgery ward until ready to go home (approximately 4 days).

CABG can also be operated “off-pump,” i.e., without using a heart-lung bypass machine. The principals and operations of off-pump CABG are similar to that of traditional CABG. Bypass grafts, such as an artery from behind the breast bone and/or veins from the legs, are used to “bypass” blood around coronary artery blockages. However, because the heart-lung bypass machine is not used, the special catheters and “cannulae” that are placed in and around the heart for a traditional CABG operation are not used. The heart continues to pump blood to the rest of the body. Surgeons must operate, e.g., perform delicate suturing, on a “beating heart”. Consequently, stabilizing devices have been used to help to limit the motion of the heart as surgeons operate. For example, a stabilizing device is placed on the surface of the heart, limiting the motion of the beating heart. After the graft vessels are sewn to the coronary arteries and the aorta, the stabilizing devices are removed.

In an embodiment of the present invention, an allograft comprising a layer of amnion is used in an off-pump CABG in the same manner as that described above for the traditional CABG.

CABG can also be a minimally invasive direct CABG. Similar to off-pump CABG, no heart-lung bypass machine is used in the minimally invasive direct CABG. Instead of a large incision to open the chest bone as in the traditional or off-pump CABG, several small incisions are made on the left side of the chest between the ribs. The minimally invasive direct CABG mainly is used for bypassing the blood vessels in front of the heart, particularly when only one or two coronary arteries need to be bypassed.

In an embodiment of the present invention, an allograft comprising a layer of amnion is used in minimally invasive direct CABG in a manner similar to that described above for the traditional CABG. The allograft is place over suture lines and also under the several small incisions of the pericardium to form a cover and barrier over the heart muscle and aorta. The layer can be placed inside the pericardium or outside the pericardium.

Amnions used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed for medical uses. The informed consent includes consent for risk assessment and consent for donation of birth tissues.

Risk assessment is conducted on a pregnant female with informed consent to evaluate her risk factors for communicable diseases, such as human immunodeficiency virus (HIV), hepatitis B virus (HBV), hepatitis C virus (HCV), cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2, HCV, HbCore, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who are tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should be used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4 to 28° C., for example, in an ice bucket.

According to an embodiment of the invention, shortly after its expulsion after birth, a suitable human placenta is placed in a sterile zip-lock plastic bag, which is placed in an ice bucket, and is delivered to another location. The placenta is rinsed, e.g., with sterile saline, to remove excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results on screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation.

The chorion can also be separated from the other tissues, preserved and stored for future use.

The isolated amnion is a tough, transparent, nerve-free and nonvascular sheet of membrane. It can be dried or lyophilized using various methods. For example, it can be dried over a sterile mesh by being placed on a sterile nitrocellulose filter paper and air dried for more than 50 minutes in a sterile environment. It can also be dried or lyophilized over other forms of supporting material, which would facilitate the subsequent manipulation of the amnion, such as sterilizing, sizing, cataloging, and shipping of the amnion.

The present invention encompasses a kit containing a plurality of allografts for improved CABG, each of the allografts has one or more layers of amnion, and instructions on how to use the allografts in CABG. The allograft can also comprise one or more layers of chorion or one or more layers of other collagen membranes of biological origin. The allograft can further comprise one or more therapeutically active agents, such as anti-microbial agents, growth enhancing agents, anti-inflammatory agents and agents which prevent scarring, adhesions and tethering of internal organs and the heart.

Preferably, at least two of the allografts in the kit have different sizes and/or thickness.

In one embodiment of the present invention, the kit includes an allograft having a thickness of about 0.02 mm to 0.10 mm, and an oval shape of about 3 cm to 9 cm in length and about 2 cm to 6 cm in width.

In another embodiment of the present invention, the kit further comprises a second allograft comprising a plurality layers of amnion, and optionally one or more layers of chorion, wherein the second allograft has a thickness of about 2 mm to 4 mm, and a rectangular shape of about 10 cm by 5 cm.

In yet another embodiment of the present invention, the kit further comprises an amniotic fluid.

Preferably, all the birth tissues in the kit, e.g., the amnion, chorion and amniotic fluid, are from the same biological source, i.e., the same pregnant woman.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A method of improving a coronary artery bypass grafting surgery in a subject, comprising applying at least one of an amniotic fluid and an allograft comprising a layer of amnion over a suture line or incision, or an otherwise damaged tissue site resulting from the coronary artery bypass grafting surgery, or over or under the pericardium membrane of the subject

2. The method of claim 1, wherein the allograft is attached to a tissue of the subject with a 4.0 suture.

3. The method of claim 1, wherein the allograft has a thickness of about 0.02 mm to 0.10 mm.

4. The method of claim 1, wherein the allograft applied over or under the pericardium membrane has an oval shape, about 3 cm to 9 cm in length and about 2 cm to 6 cm in width.

5. The method of claim 1, wherein the allograft consists of a single layer of amnion, two layers of amnion, or a layer of amnion and a layer of chorion.

6. The method of claim 1, wherein the allograft is applied after a bypass vessel graft is sutured in and the pericardial cavity is washed with a saline solution comprising one or more anti-microbial agents.

7. The method of claim 1, wherein the allograft further comprises one or more therapeutically active agents selected from the group consisting of anti-microbial agents, growth enhancing agents, anti-inflammatory agents, and agents that prevent scarring, adhesions and tethering of internal organs and the heart.

8. The method of claim 1, wherein the coronary artery bypass grafting is a traditional coronary artery bypass grafting, an off-pump coronary artery bypass grafting, or a minimally invasive direct coronary artery bypass grafting.

9. The method of claim 1, further comprising placing a second allograft comprising a layer of amnion over a harvest site for a bypass vessel graft.

10. A method of improving a coronary artery bypass grafting surgery, comprising placing an allograft comprising a layer of amnion over a harvest site for a bypass vessel graft.

11. The method of claim 10, wherein the harvest site is a left internal mammary artery (LIMA) harvest site or a saphenous vein harvest site.

12. The method of claim 10, wherein the allograft has a thickness of about 2 mm to 4 mm.

13. The method of claim 12, wherein the allograft comprises multiple layers of amnion and optionally multiple layers of chorion.

14. The method of claim 10, wherein the allograft has a rectangular shape, about 10 cm by 5 cm.

15. The method of claim 1, wherein the amnion is obtained using a process comprising:
a. obtaining informed consent from pregnant females;
b. conducting risk assessment on the consented pregnant females to select an amnion donor;
c. procuring after birth placenta from the amnion donor; and
d. obtaining the amnion from the placenta.

16. A kit comprising a plurality of allografts and instructions on how to use the allografts to improve a coronary artery bypass grafting surgery, wherein each of the plurality of allografts comprises a layer of amnion of a pre-made size and shape suitable for covering a suture line, an incision, or an otherwise damaged tissue site resulting from the coronary artery bypass grafting surgery, or for covering over or under the pericardium membrane of the subject.

17. The kit of claim 16, wherein the amnion is obtained using a process comprising:
a. obtaining informed consent from pregnant females;
b. conducting risk assessment on the consented pregnant females to select an amnion donor;
c. procuring after birth placenta from the amnion donor; and
d. obtaining the amnion from the placenta.

18. The kit of claim 16, comprising an allograft having a thickness of about 0.02 mm to 0.10 mm, and an oval shape of about 3 cm to 9 cm in length and about 2 cm to 6 cm in width.

19. The kit of claim 18, further comprising a second allograft comprising a plurality layers of amnion, and optionally one or more layers of chorion, wherein the second allograft has a thickness of about 2 mm to 4 mm, and a rectangular shape of about 10 cm by 5 cm.

20. The kit of claim 19 further comprising an amniotic fluid.
